(19) United States
(12) Patent Application Publication
(10) Pub. No.: US 2006/0166937 A1
(43) Pub. Date: Jul. 27, 2006

(54) Bisphosphonates for the treatment of atherosclerosis and device comprising them

(76) Inventor: Margaret Forney Prescott, Morris Plains, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/531,677

(22) PCT Filed: Oct. 14, 2003
(86) PCT No.: PCT/EP03/11379

§ 371 Date: Apr. 15, 2005

(30) Foreign Application Priority Data
Oct. 15, 2002 (US) 60418554
Nov. 22, 2002 (US) 60428621

(51) Int. Cl. A61K 31/675; A61K 31/663

(52) U.S. Cl. 514/89; 514/102

(57) ABSTRACT

A method for the treatment of atherosclerosis, in particular atherosclerotic/valve calcification and plaque rupture/stroke, in a patient in need of such treatment, e.g. a patient with aterial or valve calcification, which comprises administering an effective amount of a bisphosphonate, e.g. zoledronic acid or salts or hydrates thereof, to the patient. Especially the invention relates to the local administration the bisphosphonate and to a device, e.g. stent adapted for such local administration.

This invention relates to new therapeutic uses of bisphosphonates and to therapeutic devices comprising bisphosphonates.

Bisphosphonates are widely used to inhibit osteoclast activity in a variety of both benign and malignant diseases which involve excessive or inappropriate bone resorption. These pyrophosphate analogs not only reduce the occurrence of skeletal related events but they also provide patients with clinical benefit and improve survival. Bisphosphonates are able to prevent bone resorption in vivo; the therapeutic efficacy of bisphosphonates has been demonstrated in the treatment of osteoporosis, osteopenia, Paget's disease of bone, tumour-induced hypercalcemia (TIH) and, more recently, bone metastases (BM) and multiple myeloma (MM) (for review see Fleisch H 1997 Bisphosphonates clinical. In Bisphosphonates in Bone Disease. From the Laboratory to the Patient. Eds: The Parthenon Publishing Group, New York/London pp 68-163). The mechanisms by which bisphosphonates inhibit bone resorption are still not completely understood and seem to vary according to the bisphosphonates studied. Bisphosphonates have been shown to bind strongly to the hydroxyapatite crystals of bone, to reduce bone turn-over and resorption, to decrease the levels of hydroxyproline or alkaline phosphatase in the blood, and in addition to inhibit the formation, recruitment, activation and the activity of osteoclasts. Recently farnesyl diphosphate synthase, an enzyme of the mevalonate pathway of cholesterol biosynthesis, has been identified as the molecular target of nitrogen-containing bisphosphonates (reviewed in Rogers M J, Gordon S, Benford H L, Coxon F P, Luckman S P, Monkkonen J, Frith J C. 2000. Cellular and molecular mechanisms of action of bisphosphonates. Cancer 88(suppl):2961-2978).

Evidence is accumulating for a link between vascular and bone disease: Calcification being a common feature of atherosclerotic plaques, and osteoporosis being associated with both atherosclerosis and vascular calcification. A correlation has been found between coronary calcificaiton and low bone mineral density (Barengolts E I, Berman M, Kukreja S C, Kouznetsova T, Lin C, Chomka E V, Osteoporosis and coronary atherosclerosis in asymptomatic postmenopausal women. Calcif Tissue Int. 1998 62:209-13) as well as between carotid atheroscslerotic plaque extent and low bone mineral density (Uyama O, Yoshimoto Y, Yamamoto Y, Kawai A, Bone changes and carotid atherosclerosis in postmenopausal women. Stroke. 1997 28:1730-2). A longitudinal study has demonstrated that patients with the greatest magnitude of bone loss also have the most severe progresison of aortic calcification (Kiel D P, Kauppila L I, Cupples L A, Hannan M T, O'Donnell C J, Wilson P W, Bone loss and the progression of abdominal aortic calcification over a 25 year period: the Framingham Heart Study, Calcif Tissue Int. 2001 68:271-6). Furthermore, patients with Singleton-Merten syndrome exhibit both progressive osteoporosis and progressive calcification of the aorta and valves (Singleton E B, Merten D F, An unusual syndrome of widened medullary cavities of the metacarpals and phalanges, aortic calcification and abnormal dentition. Pediatr Radiol. 1973 1:2-7). Atherosclerotic lesions share features with skeletal bone including the presence of the major bone matrix proteins (osteonectin, osteocalcin, osteopontin) as well as frank bone (Doherty T M, Uzui H, Fitzpatrick L A, Tripathi P V, Dunstan C R, Asotra K, Rajavashisth T B. Rationale for the role of osteoclast-like cells in arterial calcification. FASEB J. 2002 16:577-82; Watson K E, Demer L L. The atherosclerosis-calcification link? Curr Opin Lipidol. 1996 7:101-4). However, the relationship between atherosclerosis and osteoporosis as well as the relationship of vascular calcification to the pathogenesis of atherosclerosis remains incompletely understood.

Many humans suffer from circulatory diseases caused by a progressive blockage of the blood vessels that perfuse major organs such as heart, liver, kidney and brain. Severe blockage of blood vessels in such humans often leads to e.g. ischemic injury, hypertension, stroke or myocardial infarction. Atherosclerotic lesions which limit or obstruct coronary or peripheral blood flow are the major cause of ischemic disease related morbidity and mortality including coronary heart disease, stroke, aneurysm and peripheral claudication. To stop the disease process and prevent the more advanced disease states in which the cardiac muscle or other organs are compromised, medical revascularization procedures such as percutaneous transluminal coronary angioplasty (PCTA), percutaneous transluminal angioplasty (PTA), stenting, atherectomy, and other types of catheter-based revascularization/local drug-delivery techniques at the site of disease, either applied via the vessel lumen or applied via the external/adventitial aspect of the vessel, as well as bypass grafting are used. Ultrasound-activation of drug-containing microbubbles provides a noninvasive mechanism for local drug delivery during revascularization.

In addition to the proliferative narrowing/occlusion seen in native arteries, a similar luminal compromise process with resultant obstruction/diminution of blood flow occurs within by-pass grafts, at sites of anastomoses in transplantation, in vessels used to create dialysis access, and in veins. Thus revascularization procedures such as angioplasty and/or stenting and/or other types of catheter-based local delivery are also used in these pathologic conditions.

Re-narrowing (restenosis) of an artherosclerotic coronary artery after various revascularization procedures occurs in 10-80% of patients undergoing this treatment, depending on the procedure used and the aterial site. Moreover, opening an artery obstructed by atherosclerosis, revascularization also injures endothelial cells and smooth muscle cells within the vessel wall, thus initiating a thrombotic and inflammatory response. Cell derived growth factors such as platelet derived growth factor, infiltrating macrophages, leukocytes or the smooth muscle cells themselves provoke proliferative and migratory responses in the smooth muscle cells. Simultaneously with local proliferation and migration, inflammatory cells also invade the site of vascular injury and may migrate to the deeper layers of the vessel wall. Proliferation/migration usually begins within one to two days post-injury and, depending on the revascularization procedure used, continues for days and weeks. Both cells within the atherosclerotic lesion and those within the media migrate, proliferate and/or secrete significant amounts of extracellular matrix proteins. Proliferation, migration and extracellular matrix synthesis continue until the damaged endothelial layer is repaired at which time proliferation slows within the intima. The newly formed tissue is called neointima, intimal thickening or restenotic lesion and usually results in narrowing of the vessel lumen. Further lumen narrowing may take place due to constructive remodeling, e.g. vascular remodeling, leading to further intimal thickening or hyperplasia.

Accordingly, there is a need for effective treatment and drug delivery systems for preventing and treating intimal thickening or restenosis that occurs after injury, e.g. vascular injury, including e.g. surgical injury, e.g. revascularization-induced injury, e.g. also in heart or other grafts.

Thus it is an object of this invention to provide a drug-containing medical device which allows sustained delivery of a pharmaceutical or sufficient pharmaceutical activity at or near the coated surfaces of the devices.

Also, it is an object of the invention to provide medical devices with stabilized complexed drug coatings and methods for making such devices.

Additionally, it is an object of the invention to provide a drug-releasing coated stent or medical device to allow the timed or prolonged application of the drug to body tissue. It is a further object of the invention to provide methods for making a drug-releasing medical device, which permits timed-delivery or long-term delivery of a drug. Thus, there is a need for improved bio-compatible complexed drug coatings which enhance the biostability, abrasion-resistance, lubricity and bio-activity of the surface of implantable medical devices, especially complexed drug coatings which contain heat-sensitive biomolecules. In particular, there is a need for improved, cost efficient complexed drug coatings and devices, which have anti atherosclerotic and/or antithrombogenic and/or anti-restenosis and/or anti-inflammatory properties and for more efficient methods of providing same. The present invention is directed to meeting these and other needs.

It has now been surprisingly found that bisphosphonates, in particular zoledronic acid, optionally in conjunction with other active compounds, e.g. compounds having mTOR inhibiting properties or compounds having anti-inflammatory properties, may be used for the treatment of atherosclerosis, and in particular have beneficial effects when locally applied to the Atherosclerotic lesions sites.

It has particularly been found that zoledronic acid is surprisingly well adapted for delivery especially controlled delivery from a catheter-based device, an intraluminal medical device or a device applied to the external/adventitial aspect of the vessel. The pharmaceutically acceptable polymers do not alter or adversely impact the therapeutic properties of zoledronic acid. On the contrary, zoledronic acid, is particularly stable in any pharmaceutically acceptable polymers at body temperature and in human plasma, permitting an unexpected long storage in a coated stents. zoledronic acid is particularly well adapted because it is easily secured to the medical device by the polymer and the rate at which it is released from coating to the body tissue can be easily controlled. Furthermore, zoledronic acid coated stents permit long-term delivery of the drug. It is particularly worthwhile to control the bioeffectiveness of the zoledronic acid coated stents in order to obtain the same biological effect as a liquid dosage.

Additionally, it has now been found that zoledronic acid has a beneficial effect on atherosclerosis and on restenosis and stenosis following transplantation, in particular atherosclerosis and restenosis following revascularisation.

Accordingly in a first aspect the present invention provides a method for the treatment of atherosclerosis in a patient in need of such treatment, which comprises administering an effective amount of a bisphosphonate to the patient.

In this aspect the invention further provides use of a bisphosphonate in the preparation of a medicament for the treatment of atherosclerosis.

In this aspect the invention yet further provides use of a bisphosphonate to treat atherosclerosis associated with diseases or pathological conditions in mammals.

In this aspect the invention even yet further provides use of a bisphoshonate to treat calcification associated with renal failure.

The present invention is particularly applicable to the prevention and treatment of atherosclerotic calcification of blood vessels, e.g. arteries, and valves, e.g. heart valves.

In addition to inhibiting bone resorption, bisphosphonates advantageously may also inhibit and possibly even reverse angiogenesis associated with diseases or pathological conditions in mammals (WO 00/7114). Thus bisphosphonate treatment of patients with atherosclerotic plaques may advantageously result in inhibition of angiogenesis associated with plaque instability and rupture which can result in thromboses and the like. Furthermore, inhibition of calcification, and in particular inhibition of calcified nodule formation, may inhibit or decrease plaque instability and rupture. Prog Cardiovasc Dis. 2002 44:349-56).

Thus in a particular embodiment the invention includes use of bisphosphonate treatment to stabilise atherosclerotic plaques and thus decrease the risk of myocardial infarction, sudden death and stroke.

In the present description the terms “treatment” or “treat” refer to both prophylactic or preventative treatment as well as curative or palliative treatment of atherosclerosis, in particular the prevention and treatment of atherosclerotic calcification of arteries and valves, and stabilisation of atherosclerotic plaques preferably with resultant decrease of the risk of myocardial infarction, sudden death, acute coronary syndromes, peripheral artery claudication and stroke. In addition and particulary the invention includes the treatment and prevention of smooth muscle cell proliferation and migration in hollow tubes, or increased cell proliferation or decreased apoptosis or increased matrix deposition, as well as the treatment of intimal thickening in vessel walls.

Accordingly in a further embodiment the invention provides:

(I) a method for the prevention and treatment of atherosclerotic calcification of blood vessels and valves in a patient, which comprises administering an effective amount of a bisphosphonate to the patient; and

(II) use of a bisphosphonate in the preparation of a medicament for the prevention and treatment of atherosclerotic calcification of blood vessels and valves.

(III) use of a bisphosphonate in the preperation of a medicament for the prevention and treatment of calcification of blood vessels and valves associated with renal failure.

Accordingly in a yet further embodiment the invention provides:

(I) a method for the stabilisation of atherosclerotic plaques in a patient, which comprises administering an effective amount of a bisphosphonate to the patient; and

(II) use of a bisphosphonate in the preparation of a medicament for stabilisation of atherosclerotic plaques.

Accordingly in a still yet further embodiment the invention provides:

(I) a method for the treatment and prevention of smooth muscle cell proliferation and migration in hollow tubes, or increased cell proliferation or decreased apoptosis or increased matrix deposition, or the treatment of intimal thickening in vessel walls in a patient, which comprises administering an effective amount of a bisphosphonate to the patient; and

(II) use of a bisphosphonate in the preparation of a medicament the treatment and prevention of smooth muscle cell proliferation and migration in hollow tubes, or increased cell proliferation or decreased apoptosis or increased matrix deposition, or the treatment of intimal thickening in vessel walls.

The uses and methods of the present invention represent an improvement to existing therapy of malignant diseases in which bisphosphonates are used to prevent or inhibit development of bone metastases or excessive bone resorption, and also for the therapy of inflammatory diseases such as rheumatoid arthritis and osteoarthritis, as well as for all forms of osteoporosis and osteopenia.

The bisphosphonates for use in the present invention are preferably N-bisphosphonates.

For the purposes of the present description an N-bisphosphonate is a compound which in addition to the characteristic geminal bisphosphate (P-C-P) moiety comprises a nitrogen containing side chain, e.g. a compound of formula I 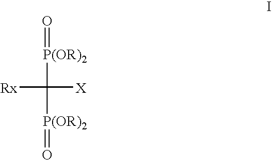
wherein
X is hydrogen, hydroxyl, amino, alkanoyl, or an amino group substituted by C₁-C₄ alkyl, or alkanoyl;
R is hydrogen or C₁-C₄ alkyl and
Rx is a hydrocarbyl side chain which contains an optionally substituted amino group, or a nitrogen containing heterocycle (including aromatic nitrogen-containing heterocycles), and pharmaceutically acceptable salts thereof or any hydrate thereof.

Thus, for example, suitable N-bisphosphonates for use in the invention may include the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methylpyridinium salts thereof, for example N-methylpyridinium iodides such as NE-10244 or NE-10446; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); and 1-hydroxy-2-(imidazo[1,2-a]pyridin-3-yl)ethane-1,1-diphosphonic acid, e.g. YM 529.

In one embodiment a particularly preferred N-bisphosphonate for use in the invention comprises a compound of Formula II 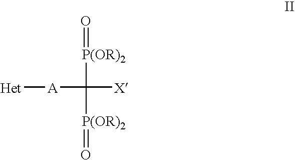
wherein
Het is an imidazole, oxazole, isoxazole, oxadiazole, thiazole, thiadiazole, pyridine, 1,2,3-triazole, 1,2,4-triazole or benzimidazole radical, which is optionally substituted by alkyl, alkoxy, halogen, hydroxyl, carboxyl, an amino group optionally substituted by alkyl or alkanoyl radicals or a benzyl radical optionally substituted by alkyl, nitro, amino or aminoalkyl;
A is a straight-chained or branched, saturated or unsaturated hydrocarbon moiety containing from 1 to 8 carbon atoms;
X′ is a hydrogen atom, optionally substituted by alkanoyl, or an amino group optionally substituted by alkyl or alkanoyl radicals, and
R is a hydrogen atom or an alkyl radical,
   and the pharmacologically acceptable salts thereof.

In a further embodiment a particularly preferred bisphosphonate for use in the invention comprises a compound of Formula III 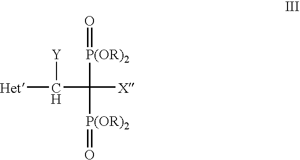
wherein
Het′ is a substituted or unsubstituted heteroaromatic five-membered ring selected from the group consisting of imidazolyl, imidazolinyl, isoxazolyl, oxazolyl, oxazolinyl, thiazolyl, thiazolinyl, triazolyl, oxadiazolyl and thiadiazolyl wherein said ring can be partly hydrogenated and wherein said substituents are selected from at least one of the group consisting of C₁-C₄ alkyl, C₁-C₄ alkoxy, phenyl, cyclohexyl, cyclohexylmethyl, halogen and amino and wherein two adjacent alkyl substituents of Het can together form a second ring;
Y is hydrogen or C₁-C₄ alkyl;
X″ is hydrogen, hydroxyl, amino, or an amino group substituted by C₁-C₄ alkyl, and
R is hydrogen or C₁-C₄ alkyl;
   as well as the pharmacologically acceptable salts and isomers thereof.

In a yet further embodiment a particularly preferred bisphosphonate for use in the invention comprises a compound of Formula IV 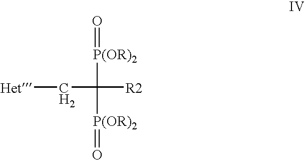
wherein
Het′″ is an imidazolyl, 2H-1,2,3-, 1H-1,2,4- or 4H-1,2,4-triazolyl, tetrazolyl, oxazolyl, isoxazolyl, oxadiazolyl, thiazolyl or thiadiazolyl radical which is unsubstituted or C-mono-or di-substituted by lower alkyl, by lower alkoxy, bx phenyl which may in turn be mnon- or disubstituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen, and
R2 is hydrogen, hydroxy, amino, lower alkylthio or halogen,
lower radicals having up to and including 7 C-atoms,
   or a pharmacologically acceptable salt thereof.

Examples of particularly preferred N-bisphosphonates for use in the invention are:
2-(1-Methylimidazol-2-yl)-1-hydroxyethane-1,1-diphosphonic acid;
2-(1-Benzylimidazol-2-yl)-1-hydroxyethane-1,1-diphosphonic acid;
2-(1-Methylimidazol-4-yl)-1-hydroxyethane-1,1-diphosphonic acid;
1-Amino-2-(1-methylimidazol-4-yl)ethane-1,1-diphosphonic acid;
1-Amino-2-(1-benzylimidazol-4-yl)ethane-1,1-diphosphonic acid;
2-(1-Methylimidazol-2-yl)ethane-1,1-diphosphonic acid;
2-(1-Benzylimidazol-2-yl)ethane-1,1-diphosphonic acid;
2-(Imidazol-1-yl)-1-hydroxyethane-1,1-diphosphonic acid;
2-(Imidazol-1-yl)ethane-1,1-diphosphonic acid;
2-(4H-1,2,4-triazol-4-yl)-1-hydroxyethane-1,1-diphosphonic acid;
2-(Thiazol-2-yl)ethane-1,1-diphosphonic acid;
2-(Imidazol-2-yl)ethane-1,1-diphosphonic acid;
2-(2-Methylimidazol-4(5)-yl)ethane-1,1-diphosphonic acid;
2-(2-Phenylimidazol-4(5)-yl)ethane-1,1-diphosphonic acid;
2-(4,5-Dimethylimidazol-1-yl)-1-hydroxyethane-1,1-diphosphonic acid, and
2-(2-Methylimidazol-4(5)-yl)-1-hydroxyethane-1,1-diphosphonic acid,
   and pharmacologically acceptable salts thereof.

The most preferred N-bisphosphonate for use in the invention is 2-(imidazol-1yl)-1-hydroxyethane-1,1-diphosphonic acid (zoledronic acid) or a pharmacologically acceptable salt thereof.

All the N-bisphosphonic acid derivatives mentioned above are well known from the literature. This includes their manufacture (see e.g. EP-A-513760, pp. 1348). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hydroxy-2-(imidazol-1-yl)ethane-1,1-diphosphonic acid is prepared as described e.g. in U.S. Pat. No. 4,939,130. See also U.S. Pat. Nos. 4,777,163, 4,687,767, and EP 0 275 821 B.

The N-bisphosphonates may be used in the form of an isomer or of a mixture of isomers where appropriate, typically as optical isomers such as enantiomers or diastereoisomers or geometric isomers, typically cis-trans isomers. The optical isomers are obtained in the form of the pure antipodes and/or as racemates.

The N-bisphosphonates can also be used in the form of their hydrates or include other solvents used for their crystallisation.

Pharmacologically acceptable salts are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

Especially preferred pharmaceutically acceptable salts are those where one, two, three or four, in particular one or two, of the acidic hydrogens of the bisphosphonic acid are replaced by a pharmaceutically acceptable cation, in particular sodium, potassium or ammonium, in first instance sodium.

A very preferred group of pharmaceutically acceptable salts is characterized by having one acidic hydrogen and one pharmaceutically acceptable cation, especially sodium, in each of the phosphonic acid groups.

The Agents of the Invention (the N-bisphosphonates) are preferably used in the form of pharmaceutical compositions that contain a therapeutically effective amount of active ingredient optionally together with or in admixture with inorganic or organic, solid or liquid, pharmaceutically acceptable carriers which are suitable for administration.

The pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

Preferably, the pharmaceutical compositions are adapted to oral or parenteral (especially intravenous, intra-arterial or transdermal) administration. Intravenous and oral, first and foremost intravenous, administration is considered to be of particular importance. Preferably the bisphosphonate active ingredient is in the form of a parenteral, most preferably an intravenous form.

The particular mode of administration and the dosage may be selected by the attending physician taking into account the particulars of the patient, especially age, weight, life style, activity level, hormonal status (e.g. post-menopausal) and bone mineral density as appropriate. Most preferably, however, the bisphosphonate is administered intravenously.

The dosage of the Agents of the Invention may depend on various factors, such as effectiveness and duration of action of the active ingredient, mode of administration, warm-blooded species, and/or sex, age, weight and individual condition of the warm-blooded animal.

Normally the dosage is such that a single dose of the bisphosphonate active ingredient from 0.002-20.0 mg/kg, especially 0.01-10.0 mg/kg, is administered to a warm-blooded animal weighing approximately 75 kg. If desired, this dose may also be taken in several, optionally equal, partial doses.

“mg/kg” means mg drug per kg body weight of the mammal—including man—to be treated.

The dose mentioned above—either administered as a single dose (which is preferred) or in several partial doses—may be repeated, for example once daily, once weekly, once every month, once every three months, once every six months or once a year. In other words, the pharmaceutical compositions may be administered in regimens ranging from continuous daily therapy to intermittent cyclical therapy.

Preferably, the bisphosphonates are administered in doses which are in the same order of magnitude as those used in the treatment of the diseases classically treated with bisphosphonic acid derivatives, such as Paget's disease, tumour-induced hypercalcemia or osteoporosis. In other words, preferably the bisphosphonic acid derivatives are administered in doses which would likewise be therapeutically effective in the treatment of Paget's disease, tumour-induced hypercalcaemia or osteoporosis, i.e. preferably they are administered in doses which would likewise effectively inhibit bone resorption. For example, for the preferred nitrogen-containing bisphosphonates, e.g. zoledronic acid and salts thereof, doses of bisphosphonate in the range from about 0.5 to about 20 mg, preferably from about 1 to about 10 mg, may be used for treatment of human patients.

Formulations in single dose unit form contain preferably from about 1% to about 90%, and formulations not in single dose unit form contain preferably from about 0.1% to about 20%, of the active ingredient. Single dose unit forms such as capsules, tablets or dragees contain e.g. from about 1 mg to about 500 mg of the active ingredient.

Pharmaceutical preparations for enteral and parenteral administration are, for example, those in dosage unit forms, such as dragees, tablets or capsules and also ampoules. They are prepared in a manner known per se, for example by means of conventional mixing, granulating, confectioning, dissolving or lyophilising processes.

For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragee cores. Suitable carriers are especially fillers, such as sugars, for example lactose, saccharose, mannitol or sorbitol, cellulose preparations and/or calcium phosphates, for example tricalcium phosphate or calcium hydrogen phosphate, and also binders, such as starch pastes, using, for example, corn, wheat, rice or potato starch, gelatin, tragacanth, methylcellulose and/or polyvinylpyrrolidone and, if desired, disintegrators, such as the above-mentioned starches, also carboxymethyl starch, crosslinked polyvinylpyrrolidone, agar or alginic acid or a salt thereof, such as sodium alginate. Adjuncts are especially flow-regulating agents and lubricants, for example silicic acid, talc, stearic acid or salts thereof, such as magnesium or calcium stearate, and/or polyethylene glycol. Dragee cores are provided with suitable coatings that may be resistant to gastric juices, there being used, inter alia, concentrated sugar solutions that optionally contain gum arabic, talc, polyvinylpyrrolidone, polyethylene glycol and/or titanium dioxide, or lacquer solutions in suitable organic solvents or solvent mixtures or, to produce coatings that are resistant to gastric juices, solutions of suitable cellulose preparations, such as acetylcellulose phthalate or hydroxypropylmethylcellulose phthalate. Colouring substances or pigments may be added to the tablets or dragee coatings, for example for the purpose of identification or to indicate different doses of active ingredient.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intra-arterially, intramuscularly, intraperitoneally, intranasally, intradermally, subcutaneously or preferably intravenously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

In a further aspect in accordance with the present invention, the bisphosphonate is locally applied to the atherosclerosis lesion site, especially by controlled delivery from a catheter-based device, an intraluminal medical device or a device applied to the external/adventitial aspect of the blood vessel.

It has been found that circulatory diseases caused by a progressive blockage of the blood vessels that perfuse major organs such as heart, liver, kidney and brain can be treated by applying a drug delivery device to the external/adventitial aspect of the vessel, e.g. wrapped around the vessel, which then release a drug that is anti atherosclerotic and/or antithrombogenic and/or anti-restenosis and/or anti-inflammatory.

In the present description hollow tube is intended to mean any hollow tube that has the function of transporting a gas or liquid, preferably a liquid and most preferably blood for example a vessel, vein, artery etc. and that can be affected by atherosclerosis and/or thrombogenosis and/or restenosis and/or inflammation.

In the present description references to bisphosphonate, e.g. zoledronic acid, include the pharmaceutically salts and esters thereof in addition to the free bisphosphonic acid, and that salts and acids include hydrates thereof as well as solvates.

According to the invention, the bisphosphonate, e.g. zoledronic acid, may be applied as the sole active ingredient or in conjunction with an immunosuppressive agent, e.g. a calcineurin inhibitor, e.g. a cyclosporin, for example cyclosporin A, or FK506, an EDG-Receptor agonist, e.g. FTY720, a mTOR inhibitor agent e.g. rapamycin derivatives, e.g. 40-O-(2-hydroxyethyl)-rapamycin, an anti-inflammatory agent, e.g. a steroid, e.g. a corticosteroid, e.g. dexamethasone or prednisone, an NSAID, e.g. a cyclooxygenase inhibitor, e.g. a cox-2 inhibitor, e.g. celecoxib, rofecoxib, etoricoxib, valdecoxib or lumiracoxib, or an antiproliferative agent, e.g. a microtubule stabilizing or destabilizing agent including but not limited to taxanes, e.g. taxol, paclitaxel or docetaxel, vinca alkaloids, e.g. vinblastine, especially vinblastine sulfate, vincristine especially vincristine sulfate, and vinorelbine, discodermolides or epothilones or a derivative thereof, e.g. epothilone B or a derivative thereof or a telomerase inhibitior, a tyrosine kinase inhibitor, e.g. staurosporin and related small molecules, e.g. UCN-01, BAY 43-9006, Bryostatin 1, Perifosine, Limofosine, midostaurin, RO318220, RO320432, GO 6976, Isis 3521, LY333531, LY379196, SU5416, SU6668, AG1296, agents generically termed tyrphostins such as AG957.

In this further aspect the present invention also provides the local administration or delivery of a bisphosphonate, e.g. zoledronic acid, in conjunction with a calcineurin inhibitor, e.g. as disclosed above, a mTOR inhibitor agent e.g. rapamycin derivatives, e.g. 40-O-(2-hydroxyethyl)-rapamycin, an EDG-Receptor agonist, e.g. as disclosed above, a microtubule stabilizing or destabilizing agent, e.g. as disclosed above, an osteoclast activity inhibitor, e.g. as disclosed above, a compound or antibody which inhibits the PDGF receptor tyrosine kinase or a compound which binds to PDGF or reduces expression of the PDGF receptor, e.g. as disclosed above, a compound or antibody which inhibits the EGF receptor tyrosine kinase or a compound which binds to EGF or reduces expression of the EGF receptor, e.g. as disclosed above, a compound or antibody which inhibits the VEGF receptor tyrosine kinase or a VEGF receptor or a compound which binds to VEGF, e.g. as disclosed above, an inhibitor of a modulator (i.e. antagonists or agonists) of kinases, e.g. as disclosed above.

In accordance with the particular findings of this further aspect the present invention, there is provided:
1. A method for preventing or treating smooth muscle cell proliferation and migration in hollow tubes, or increased cell proliferation or decreased apoptosis or increased matrix deposition in a mammal in need thereof, comprising local administration of a therapeutically effective amount of a bisphosphonate, e.g. zoledronic acid, optionally in conjunction with one or more other active ingredients, e.g. as disclosed above.
2. A method for the stabilisation of vulnerable atherosclerotic plaques in a mammal in need thereof, comprising local administration of a therapeutically effective amount of a bisphosphonate, e.g. zoledronic acid, optionally in conjunction with one or more other active ingredients, e.g. as disclosed above.
3. A method for the treatment of intimal thickening in vessel walls or stabilisation of vulnerable atherosclerotic plaques comprising the controlled delivery from any catheter-based device, intraluminal medical device or device applied to the externally adventitial aspect of the vessel of a therapeutically effective amount of a bisphosphonate, e.g. zoledronic acid, optionally in conjunction with one or more other active ingredients, e.g. as disclosed above.
4. A drug delivery device or system comprising a) a medical device adapted for local application or administration in hollow tubes, e.g. a catheter-based delivery device, intraluminal medical device or a device applied to the external/adventitial aspect of the vessel, and b) a therapeutic dosage of a bisphosphonate, e.g. zoledronic acid, optionally in conjunction with a therapeutic dosage of one or more other active ingredients, e.g. as disclosed above, each being releasably affixed to the catheter-based delivery device, medical device or a device applied to the external/adventitial aspect of the vessel.

In preferred embodiments the invention includes methods of treatment, uses and devices for:
a) Intimal thickening in vessel walls due to atherosclerosis, stenosis or restenosis, e.g. following revascularization, neovascularization or any other vascular procedure besides traditional revascularisation, and/or inflammation and/or thrombosis.
b) Closure of A-V access e.g. the access used in renal dialysis patients
c) Constrictive remodeling proximal and distal to the catheter-based device or intraluminal medical device
d) Vulnerable atherosclerotic plaques.

Such a local delivery device or system can be used to reduce atherosclerosis, stenosis or restenosis as an adjunct to revascularization, bypass or grafting procedures or any other vascular procedure besides traditional revascularisation e.g. CABG, aneurysm repair, anastomic hyperplasia, performed in any vascular location including coronary arteries, carotid arteries, renal arteries, peripheral arteries, cerebral arteries or any other arterial or venous location, to reduce anastomic stenosis such as in the case of arterial-venous dialysis access with or without polytetrafluoroethylene grafting and with or without stenting, or in in conjunction with any other heart or transplantation procedures, or congenital vascular interventions. Use to reduce, constrictive remodeling proximal and distal to the drug delivery device or system, reduced flow after any other vascular procedure besides traditional revascularisation e.g. CABG, aneurysm repair, anastomic hyperplasia, local delivery to the adventitia accompanying revascularisation, etc., closure of A-V access e.g. the access used in renal dialysis patients. Such a local delivery device or system can also be used to stabilise vulnerable atherosclerotic plaques.

A bisphosphonate, e.g. zoledronic acid, or a pharmaceutically acceptable salt thereof will be referred to hereinafter as “drug”. The other active ingredients which may be used in conjunction with zoledronic acid as disclosed above, will be referred to hereinafter collectively as “adjunct”. Drug(s) shall mean drug or drug+adjunct.

The local administration preferably takes place at or near the vascular lesions sites.

The administration may be by one or more of the following routes: via catheter or other intravascular delivery system, intranasally, intrabronchially, interperitoneally or eosophagal. Hollow tubes include circulatory system vessels such as blood vessels (arteries or veins), tissue lumen, lymphatic pathways, digestive tract including alimentary canal, respiratory tract, excretory system tubes, reproductive system tubes and ducts, body cavity tubes, etc. Local administration or application of the drug(s) affords concentrated delivery of said drug(s), achieving tissue levels in target tissues not otherwise obtainable through other administration route. Means for local drug(s) delivery to hollow tubes can be by physical delivery of the drug(s) either internally or externally to the hollow tube. Local drug(s) delivery includes catheter delivery systems, local injection devices or systems or indwelling devices. Such devices or systems would include, but not be limited to, stents, coated stents, endolumenal sleeves, stent-grafts, liposomes, controlled release matrices, polymeric endoluminal paving, or other endovascular devices, embolic delivery particles, cell targeting such as affinity based delivery, internal patches around the hollow tube, external patches around the hollow tube, hollow tube cuff, external paving, external stent sleeves, and the like. See, Eccleston et al. (1995) Interventional Cardiology Monitor 1:33-40-41 and Slepian, N. J. (1996) Intervente. Cardiol. 1:103-116, or Regar E, Sianos G, Serruys P W. Stent development and local drug delivery. Br Med Bull 2001, 59:227-48 which disclosures are herein incorporated by reference.

By “biocompatible” is meant a material which elicits no or minimal negative tissue reaction including e.g. thrombus formation and/or inflammation.

Delivery or application of the drug(s) can occur using stents or sleeves or sheathes. An intraluminal stent composed of or coated with a polymer or other biocompatible materials, e.g. porous ceramic, e.g. nanoporous ceramic, into which the drug(s) has been impregnated or incorporated may be used. Such stents may be biodegradable or may be made of metal or alloy, e.g. Ni and Ti, or another stable substance when intented for permanent use. The drug(s) may also be entrapped into the metal of the stent or graft body which has been modified to contain micropores or channels. Also lumenal and/or ablumenal coating or external sleeve made of polymer or other biocompatible materials, e.g. as disclosed above, that contain the drug(s) may also be used for local delivery.

Stents are commonly used as a tubular structure left inside the lumen of a duct or vessel to relieve an obstruction. They may be inserted into the duct lumen in a non-expanded form and are then expanded autonomously (self-expanding stents) or with the aid of a second device in situ, e.g. a catheter-mounted angioplasty balloon which is inflated within the stenosed vessel or body passageway in order to disrupt the obstructions associated with the wall components of the vessel and to obtain an enlarged lumen.

For example, the drug(s) may be incorporated into or affixed to the stent in a number of ways and utilizing any biocompatible materials; it may be incorporated into e.g. a polymer or a polymeric matrix and sprayed onto the outer surface of the stent. A mixture of the drug(s) and the polymeric material may be prepared in a solvent or a mixture of solvents and applied to the surfaces of the stents also by dip-coating, brush coating and/or dip/spin coating, the solvent (s) being allowed to evaporate to leave a film with entrapped drug(s). In the case of stents where the drug(s) is delivered from micropores, struts or channels, a solution of a polymer may additionally be applied as an outlayer to control the drug(s) release; alternatively, the drug may be comprised in the micropores, struts or channels and the adjunct may be incorporated in the outlayer, or vice versa. The drug may also be affixed in an inner layer of the stent and the adjunct in an outer layer, or vice versa. The drug(s) may also be attached by a covalent bond, e.g. esters, amides or anhydrides, to the stent surface, involving chemical derivatization. The drug(s) may also be incorporated into a biocompatible porous ceramic coating, e.g. a nanoporous ceramic coating.

Examples of polymeric materials include biocompatible degradable materials, e.g. lactone-based polyesters or copolyesters, e.g. polylactide; polylactide-glycolide; polycaprolactone-glycolide; polyorthoesters; polyanhydrides; polyaminoacids; polysaccharides; polyphosphazenes; poly(ether-ester) copolymers, e.g. PEO-PLLA, or mixtures thereof; and biocompatible non-degrading materials, e.g. polydimethylsiloxane; poly(ethylene-vinylacetate); acrylate based polymers or coplymers, e.g. polybutylmethacrylate, poly(hydroxyethyl methylmethacrylate); polyvinyl pyrrolidinone; fluorinated polymers such as polytetrafluoethylene; cellulose esters.

When a polymeric matrix is used, it may comprise 2 layers, e.g. a base layer in which the drug(s) is/are incorporated, e.g. ethylene-co-vinylacetate and polybutylmethacrylate, and a top coat, e.g. polybutylmethacrylate, which is drug(s)-free and acts as a diffusion-control of the drug(s). Alternatively, the drug may be comprised in the base layer and the adjunct may be incorporated in the outlayer, or vice versa. Total thickness of the polymeric matrix may be from about 1 to 200 or greater.

According to the method of the invention or in the device or system of the invention, the drug(s) may elute passively, actively or under activation, e.g. light-activation.

The drug(s) elutes from the polymeric material or the stent over time and enters the surrounding tissue, e.g. over a period of up to ca. 1 month to 1 year. The local delivery according to the present invention allows for high concentration of the drug(s) at the disease site with low concentration of circulating compound. The amount of drug(s) used for local delivery applications will vary depending on the compounds used, the condition to be treated and the desired effect. For purposes of the invention, a therapeutically effective amount will be administered. By therapeutically effective amount is intended an amount sufficient for therapy, e.g. to inhibit cellular proliferation and/or stabilise vulnerable atherosclerotic plaque and resulting in the prevention and treatment of the disease state. Specifically, for the prevention or treatment of atherosclerosis or restenosis e.g. after revascularization, or antitumor treatment, or vulnerable atherosclerotic plaque, local delivery may require less compound than systemic administration.

Preferred combinations are those comprising a bisphosphonate, e.g. zoledronic acid, in conjunction or association with a compound having antiproliferative properties, e.g. taxol, paclitaxel, docetaxel, an epothilone, a tyrosine kinase inhibitor, a VEGF receptor tyrosine kinase inhibitor, a VEGF receptor inhibitor, a compound binding to VEGF, a mTOR inhibitor agent e.g. rapamycin derivatives, e.g. 40-O-(2-hydroxyethyl)-rapamycin, a compound having anti-inflammatory properties, e.g. a steroid, a cyclooxygenase inhibitor. Combination of a bisphosphonate, e.g. zoledronic acid, with a compound having anti-inflammatory properties has particularly beneficial effects when used in the treatment or prevention of restenosis in diabetic patients.

Utility of the drug(s) may be demonstrated in animal test methods as well as in the clinic, for example in accordance with the methods hereinafter described.

The following Examples illustrate the invention described hereinbefore.

In the following Examples 1 to 4 the term “active ingredient” is to be understood as being any one of the bisphosphonic acid derivatives mentioned above as being useful according to the present invention.

### EXAMPLES

### Example 1

Capsules containing coated pellets of active ingredient for example, disodium pamidronate pentahydrate, as active ingredient:

Core Pellet:

| | |
|---|---|
| active ingredient (ground) | 197.3 mg |
| Microcrystalline cellulose (Avicel ® PH 105) | 52.7 mg |
| | 250.0 mg |
| +Inner coating: | |
| Cellulose HP-M 603 | 10.0 mg |
| Polyethylene glycol | 2.0 mg |
| Talc | 8.0 mg |
| | 270.0 mg |
| +Gastric juice-resistant outer coating: | |
| Eudragit ® L 30 D (solid) | 90.0 mg |
| Triethyl citrate | 21.0 mg |
| Antifoam ® AF | 2.0 mg |
| Water | |
| Talc | 7.0 mg |
| | 390.0 mg |

A mixture of active ingredient, e.g. disodium pamidronate, with Avicel® PH 105 is moistened with water and kneaded, extruded and formed into spheres. The dried pellets are then successively coated in the fluidized bed with an inner coating, consisting of cellulose HP-M 603, polyethylene glycol (PEG) 8000 and talc, and the aqueous gastric juice-resistant coat, consisting of Eudragit® L 30 D, triethyl citrate and Antifoam® AF. The coated pellets are powdered with talc and filled into capsules (capsule size 0) by means of a commercial capsule filling machine, for example Höfliger and Karg.

### Example 2

Monolith adhesive transdermal system, containing as active ingredient, for example, 1-hydroxy-2-(imidazol-1-yl)-ethane-1,1-diphosphonic acid:

Composition:

| | |
|---|---|
| polyisobutylene (PIB) 300 (Oppanol B1, BASF) | 5.0 g |
| PIB 35000 (Oppanol B10, BASF) | 3.0 g |
| PIB 1200000 (Oppanol B100, BASF) | 9.0 g |
| hydrogenated hydrocarbon resin (Escorez 5320, Exxon) | 43.0 g |
| 1-dodecylazacycloheptan-2-one (Azone, Nelson | 20.0 g |
| Res., Irvine/CA) | |
| active ingredient | 20.0 g |
| Total | 100.0 g |

Preparation:

The above components are together dissolved in 150 g of special boiling point petroleum fraction 100-125 by rolling on a roller gear bed. The solution is applied to a polyester film (Hostaphan, Kalle) by means of a spreading device using a 300 mm doctor blade, giving a coating of about 75 g/m². After drying (15 minutes at 60° C.), a silicone-treated polyester film (thickness 75 mm, Laufenberg) is applied as the peel-off film. The finished systems are punched out in sizes in the wanted form of from 5 to 30 cm² using a punching tool. The complete systems are sealed individually in sachets of aluminised paper.

### Example 3

Vial containing 1.0 mg dry, lyophilized 1-hydroxy-2-(imidazol-1-yl)ethane-1,1-diphosphonic acid (mixed sodium salts thereof). After dilution with 1 ml of water, a solution (concentration 1 mg/ml) for i.v. infusion is obtained.

Composition:

| active ingredient (free diphosphonic acid) | | 1.0 | mg |
|---|---|---|---|
| mannitol | | 46.0 | mg |
| Trisodium citrate × 2H₂O | ca. | 3.0 | mg |

| | | |
|---|---|---|
| water | 1 | ml |
| water for injection | 1 | ml. |

In 1 ml of water, the active ingredient is titrated with trisodium citrate×2H₂O to pH 6.0. Then, the mannitol is added and the solution is lyophilized and the lyophilisate filled into a vial.

### Example 4

Ampoule containing active ingredient, for instance disodium pamidronate pentahydrate dissolved in water. The solution (concentration 3 mg/ml) is for i.v. infusion after dilution.

Composition:

| | | |
|---|---|---|
| active ingredient | 19.73 | mg |
| ( [symbol] 5.0 mg of anhydrous active ingredient) | | |
| mannitol | 250 | mg |
| water for injection | 5 | ml. |

### Example 5

### The Effect of Bisphosphonates in Models of Arterial and Valve Calcification

The Effect of a Bisphosphonate on Arterial Calcification, Valve Calcification and Plaque Stabilization in a Mouse Model

A mouse model of progression of advanced atherosclerosis with development of vulnerable plaques was employed. Male, 4-6 week old LDLr−/− mice were divided into treatment groups of 18-20 animals each. All animals were fed a diet containing 1.25% cholesterol, 7.5% cocoa butter, 7.5% casein, and 0.5% sodium cholate (high cholesterol/cholic acid diet) that had previously been shown to produce extensive atheroslerosis, At 15 weeks, one group of was sacrificed to serve as pretreatment, baseline controls. The remaining LDLr−/− animals were dosed subcutaneously once per day with either vehicle or zoledronic acid solution (10 mg/kg/day) five out of seven days a week from week from week 15 through week 18 of diet administration. From weeks 19 through 22, no treatment was administered, however mice remained on the atherosclerosis-inducing high cholesterol/cholic acid diet. Mice were sacrificed at the end of week 22 and the heart and aorta were removed. The upper portion of the heart attached to the most proximal portion of the aorta containing the aortic sinus were embedded in OCT and frozen. 10 micron-thick sections were cut through the aortic sinus using a cryostat. Twenty-eight sections per animal were stained with von Kassa and/or hematoxylin. Cross-sectional areas were analyzed by planimetry to allow determination of atherosclerotic lesion area and extent of calcification within the heart valves. The remaining aorta, from the ascending aorta to the iliac bifurcation, as well as the major branches of the aorta were fixed, parafin embedded and cross sectioned for histologic analysis. Atherosclerosis extent and lesion composition was determined for the aorta and innominate (also called brachiocephalic) arteries using approriate staining methodology including hemotoxylin and eosin staining, Verhoff von Giessen staining for elastin, Von Kassa staining for calcium, and immunohistochemical stains for inflammatory cells. Four weeks of treatment with zoledronic acid significantly reduced progression of atherosclerosis in the aorta and innominate arteries between 15 and 22 weeks of high cholesterol/cholic acid diet in comparison to vehicle-treated animals. Furthermore, treatment with zoledronic acid resulted in conversion of the so-called “vulnerable” or rupture-prone plaques with a large, often calcified, lipid-rich cores and thin fibrous caps with inflammatory cell infiltration observed both at baseline and in vehicle treated animals at 22 weeks, to a more stable phenotype. At 22 weeks, animals previously treated with four weeks of zoledronic acid exhibited atherosclerotic lesions with thicker fibrous caps and less inflammatory cells and areas of calcification compared both to baseline lesions at 15 weeks and vehicle treated animals at 22 weeks. Treatment with zoledronic acid also resulted in significantly less calcification of the aortic valves compared with placebo-treated animals.

The Effect of a Bisphosphonate on Arterial and Valve Calcification in a Renal Dialysis Patient

A 52 year old male patient with end stage renal failure undergoing dialysis was diagnosed with severe osteoporosis. The patient was treated with 90 mg of pamidronate disodium administered via a 2-hour intravenous infusion once a month for a total of three months. A noticeable reduction of arterial and valve calcification was observed one year following the initial treatment with pamidronate disodium. This reduction in calcification was independently corraborated by three physicians who viewed the radiographic images in a blinded fashion.

### Example 6

### Inhibition of Late Neointimal Lesion Formation in the 28 Day Rat Carotid Artery Balloon Injury Model

Numerous compounds have been shown to inhibit intimal lesion formation at 2 weeks in the rat ballooned carotid model, while only few compounds prove effective at 4 weeks. Zoledronic acid is tested in the following rat model.

Rats are dosed orally with placebo zoledronic acid. Daily dosing starts 3 days prior to surgery and continues for 31 days. Rat carotid arteries are balloon injured using a method described by Clowes et al. Lab. Invest. 1983; 49; 208-215. Following sacrifice at 28 days post-balloon injury, carotid arteries are removed and processed for histologic and morphometric evaluation. In this assay zoledronic acid, significantly reduce neointimal lesion formation at 28 days following balloon injury when administered at a dose of from 0.2 to 3.5 mg preferably 0.5 to 2.0 mg/kg. For example, for zoledronic acid administered at 0.5, 1.0, and 2.0 mg/kg (oral gavage), the percent inhibition is similar at all three doses: inhibition is 17% at the lowest dose (0.5 mg/kg) and 37% at the highest dose (2.0 mg/kg). zoledronic acid has the beneficial effect of inhibiting lesions at 4 weeks post-ballooning

### Example 7

### Inhibition of Restenosis at 28 Days in the Rabbit Iliac Stent Model

A combined angioplasty and stenting procedure is performed in New Zealand White rabbit iliac arteries. Iliac artery balloon injury is performed by inflating a 3.0×9.0 mm angioplasty balloon in the mid-portion of the artery followed by “pull-back” of the catheter for 1 balloon length. Balloon injury is repeated 2 times, and a 3.0×12 mm stent is deployed at 6 atm for 30 seconds in the iliac artery. Balloon injury and stent placement is then performed on the contralateral iliac artery in the same manner. A post-stent deployment angiogram is performed. All animals receive oral aspirin 40 mg/day daily as anti-platelet therapy and are fed standard low-cholesterol rabbit chow. Twenty-eight days after stenting, animals are anesthetized and euthanized and the arterial tree is perfused at 100 mmHg with lactated Ringer's for several minutes, then perfused with 10% formalin at 100 mmHg for 15 minutes. The vascular section between the distal aorta and the proximal femoral arteries is excised and cleaned of periadventitial tissue. The stented section of artery is embedded in plastic and sections are taken from the proximal, middle, and distal portions of each stent. All sections are stained with hematoxylin-eosin and Movat pentachrome stains. Computerized planimetry is performed to determine the area of the internal elastic lamina (IEL), external elastic lamina (EEL) and lumen. The neointima and neointimal thickness is measured both at and between the stent struts. The vessel area is measured as the area within the EEL. Data are expressed as mean±SEM. Statistical analysis of the histologic data is accomplished using analysis of variance (ANOVA) due to the fact that two stented arteries are measured per animal with a mean generated per animal. A P<0.05 is considered statistically significant.

Zoledronic acid is administered orally by gavage at 30 mg/kg once daily from 3 days prior to stenting until day 27 post-stenting. In this model, the treatment with zoledronic acid results in a marked reduction in the extent of restenotic lesion formation compared with placebo treatment: for example, the treatment with zoledronic acid produces a significant reduction in average neointimal thickness (29% reduction; P<0.0001), neointimal area (17% reduction P<0.04), and percent arterial stenosis (17% reduction P<0.0002). Treatment with zoledronic acid did not result in differences in EEL area compared with control, indicating that treatment was not associated with either constrictive remodeling or aneurysmal-type arterial expansion. There is extensive neointimal formation in placebo-treated animals at 28 days, with the lesions consisting of abundant smooth muscle cells in proteoglycan/collagen matrix and apparent full endothelial healing. In arterial segments from the animals treated with zoledronic acid, the intima is well healed, characterized by a compact neointima consisting of smooth muscle cells and endothelium fully covering the lumen surface both over stent struts and between struts.

Stent Preparation.

A stent is weighed and then mounted for coating. While the stent is rotating, a solution of polylactide glycolide (0.70 mg/ml), zoledronic acid (0.0015 mg/ml) and tyrosine kinase inhibitor (1 mg/ml) dissolved in a mixture of methanol and tetrahydrofuran, is sprayed onto it. The coated stent is removed from the spray and allowed to air-dry. After a final weighing the amount of coating on the stent is determined.

### Example 8

### Zoledronic Acid Stability in Pharmaceutically Acceptable Polymers at Body Temperature and Zoledronic Acid Release from Polymer Coatings

Four 2 cm pieces of coated stents as described above are placed into 100 mL of phosphate buffer solution (PBS) having a pH of 7.4. Another 4 pieces from each series are placed into 100 mL of polyethylene glycol (PEG)/water solution (40/60 v/v, MW of PEG=400). The stent pieces are incubated at 37° C. in a shaker. The buffer and PEG solutions are changed daily and different assays are performed on the solutions to determine the released zoledronic acid concentrations. Such assays show a stable zoledronic acid release from coated stents for more than 45 days. By the term “stable zoledronic acid release” we mean less than 10% of variation of the drug release rate. Controlled release techniques used by the person skilled in the art allow an unexpected easy adaptation of the required zoledronic acid release rate. Thus, by selecting appropriate amounts of reactants in the coating mixture it is possible to easily control the bioeffectiveness of the zoledronic acid coated stents. Depending on the kind of coating technology used, the drug may be eluted from coating passively, actively or by light activation.

Release of zoledronic acid in plasma is also studied. 1 cm pieces of a coated stent are put into 1 mL of citrated human plasma (from Helena Labs.), which is in lyophilized form and is reconstituted by adding 1 mL of sterile deionized water. Three sets of stent plasma solutions are incubated at 37° C. and the plasma is changed daily. In a separate study, it was found that zoledronic acid in human plasma was stable at 37° C. for 72 hours. PDGF-stimulated receptor tyrosine kinase assay is performed on the last piece of each sample to determine the zoledronic acid activity. The inhibition of PDGF-stimulated receptor tyrosine kinase activity in vitro is measured in PDGF receptor immunocomplexes of BALB/c 3T3 cells, analogously to the method described by E. Andrejauskas-Buchdunger and U. Regenass in Cancer Research 52, 5353-5358 (1992). Such assays show that the activity of zoledronic acid released from stent after 45 days is still 91% of that of the normal activity of zoledronic acid. In the same assay, free zoledronic acid shows a strong decrease of its activity day after day. These assays show the unexpected high stability of zoledronic acid in polymer coatings.

### Example 9

### Examples of Synergic Combinations

Further experiments similar to that of example 6 reveal synergic combinations when zoledronic acid is used in conjunction with various other agents.

Data points just spanning the IC50 of the agents alone or in combination are entered into the CalcuSyn program (CalcuSyn, Biosoft, Cambridge UK). This program calculates a non-exclusive combination index (CI), whose value is indicative of the interaction of the two compounds, where CI˜1 represents nearly additive effects; 0.85-0.9 indicates a slight synergism and a value below 0.85 indicates synergy.

1. A method for the treatment of atherosclerosis in a patient in need of such treatment which comprises administering an effective amount of a bisphosphonate to the patient.

2. (canceled)

3. (canceled)

4. (canceled)

5. A method for the prevention and treatment of atherosclerotic calcification of blood vessels and valves in a patient, which comprises administering an effective amount of a bisphosphonate to the patient.

6. A method for the stabilisation of atherosclerotic plaques in a patient, which comprises administering an effective amount of a bisphosphonate to the patient.

7. A method for preventing or treating smooth muscle cell proliferation and migration in hollow tubes, or increased cell proliferation or decreased apoptosis or increased matrix deposition in a mammal in need thereof, comprising administration of a therapeutically effective amount of a bisphosphonate or a pharmaceutically acceptable salt thereof, optionally in conjunction with one or more other active ingredients.

8. A method for the treatment of intimal thickening in vessel walls comprising administration of a therapeutically effective amount of a bisphosphonate or a pharmaceutically acceptable salt thereof, optionally in conjunction with one or more other active ingredients

9. A method or use according to claim 1 in which the bisphonate is administered locally

10. A method for the treatment of intimal thickening in vessel walls or stabilisation of vulnerable atherosclerotic plaques comprising the controlled delivery from a catheter-based device, intraluminal medical device or device applied to the external/adventitial aspect of the vessel of a therapeutically effective amount of a bisphosphonate or a pharmaceutically acceptable salt thereof, optionally in conjunction with one or more other active ingredients

11. A method according to claim 8 wherein the bisphosphonate or a pharmaceutically acceptable salt thereof is administered or delivered in conjunction with one or more other active ingredients selected from the group consisting of a calcineurin inhibitor, an EDG-Receptor agonist, an anti-inflammatory agent, a mTOR inhibitor agent, an antiproliferative agent, a microtubule stabilizing or destabilizing agent, a tyrosine kinase inhibitor, a compound which inhibits osteoclast activity, a compound which inhibits the PDGF receptor tyrosine kinase, a compound or antibody which binds to PDGF, a compound or antibody which reduces expression of the PDGF receptor, a compound or antibody which inhibits the EGF receptor tyrosine kinase, of a compound which binds to EGF, a compound which reduces expression of the EGF receptor, a compound or antibody which inhibits the VEGF receptor tyrosine kinase or a VEGF receptor, a compound or antibody which binds to VEGF, and a modulator of kinases.

12. A drug delivery device or system comprising a) a medical device adapted for local application or administration in hollow tubes and b) a therapeutic dosage of zoledronic acid or a pharmaceutically acceptable salt thereof being releasably affixed to the medical device.

13. A device according to claim 12 comprising b) a therapeutic dosage of a bisphosphonate or a pharmaceutically acceptable salt thereof in conjunction with a therapeutic dosage of one or more other active ingredients, each being releasably affixed to the medical device and the other active ingredient being selected from the group consisting of a calcineurin inhibitor, an EDG-Receptor agonist, an anti-inflammatory agent, a mTOR inhibitor agent, an antiproliferative agent, a microtubule stabilizing or destabilizing agent, a tyrosine kinase inhibitor, a compound which inhibits osteoclast activity, a compound which inhibits the PDGF receptor tyrosine kinase, a compound or antibody which binds to PDGF, a compound or antibody which reduces expression of the PDGF receptor, a compound or antibody which inhibits the EGF receptor tyrosine kinase, a compound which binds to EGF, a compound which reduces expression of the EGF receptor, a compound or antibody which inhibits the VEGF receptor tyrosine kinase or a VEGF receptor, a compound or antibody which binds to VEGF, and a modulator of kinases.

14. A device according to claim 12 comprising b) a therapeutic dosage of a bisphosphonate or a pharmaceutically acceptable salt thereof in conjunction with a therapeutic dosage of one or more other active ingredients, each being releasably affixed to the medical device and the other active ingredient being selected from the group consisting of a calcineurin inhibitor, a mTOR inhibitor agent, an EDG-Receptor agonist, an anti-inflammatory agent, a microtubule stabilizing or destabilizing agent, a compound which inhibits osteoclast activity, a compound or antibody which inhibits the PDGF receptor tyrosine kinase, a compound which binds to PDGF or reduces expression of the PDGF receptor, a compound or antibody which inhibits the EGF receptor tyrosine kinase, a compound which binds to EGF or reduces expression of the EGF receptor, a compound or antibody which inhibits the VEGF receptor tyrosine kinase, a VEGF receptor or a compound which binds to VEGF, and an inhibitor of a modulator of kinases.

15. A method according to claim 8 wherein the administration or delivery is made using a catheter delivery system, a device applied to the external/adventitial aspect of the vessel a local injection device, an indwelling device, a stent, a coated stent, a sleeve, a stent-graft, polymeric endoluminal paving or a controlled release matrix.

16. A method according to claim 1, in which the bisphosphonate is selected from the following group of compounds or a pharmaceutically acceptable salt thereof, or any hydrate thereof: 3-amino-1-hydroxypropane-1,1-diphosphonic acid (pamidronic acid), 3-(N,N-dimethylamino)-1-hydroxypropane-1,1-diphosphonic acid, 4-amino-1-hydroxybutane-1,1-diphosphonic acid; 1-hydroxy-ethidene-bisphosphonic acid, 1-hydroxy-3-(methylpentylamino)-propylidene-bisphosphonic acid, ibandronic acid, 6-amino-1-hydroxyhexane-1,1-diphosphonic acid, 3-(N-methyl-N-n-pentylamino)-1-hydroxypropane-1,1-diphosphonic acid, 1-hydroxy-2-(imidazol-1-yl)ethane-1,1-diphosphonic acid; 1-hydroxy-2-(3-pyridyl)ethane-1,1-diphosphonic acid, including N-methylpyridinium salts thereof, 1-(4-chlorophenylthio)methane-1,1-diphosphonic acid; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, 1-(N-phenylaminothiocarbonyl)methane-1,1-diphosphonic acid, 5-benzoyl-3,4-dihydro-2H-pyrazole-3,3-diphosphonic acid tetraethyl ester, 1-hydroxy-2-(imidazo[1,2-a]pyridin-3-yl)ethane-1,1-diphosphonic acid, and 1,1-dichloromethane-1,1-diphosphonic acid.

17. A method according to claim 1, in which the bisphosphonate is a compound of Formula III 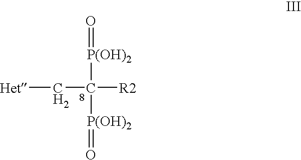
wherein
Het″ is an imidazolyl, 2H-1,2,3-, 1H-1,2,4- or 4H-1,2,4-triazolyl, tetrazolyl, oxazolyl, isoxazolyl, oxadiazolyl, thiazolyl or thiadiazolyl radical which is unsubstituted or C-mono-or di-substituted by lower alkyl, by lower alkoxy, bx phenyl which may in turn be mnon- or disubstituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen, and
R₂ is hydrogen, hydroxy, amino, lower alkylthio or halogen,
lower radicals having up to and including 7 C-atoms,
or a pharmacologically acceptable salt thereof.

18. A method according to claim 17, in which the bisphosphonate is zoledronic acid, or a pharmaceutically acceptable salt thereof, or any hydrate thereof.
